(19) United States
(12) Patent Application Publication
(10) Pub. No.: US 2012/0289612 A1
(43) Pub. Date: Nov. 15, 2012

(54) Composite Material and Method for Producing the Same

(75) Inventors: Tadashi Hamajima, Osaka (JP); Masaki Nakamura, Osaka (JP)

(73) Assignee: HOUSE FOODS CORPORATION, Osaka (JP)

(21) Appl. No.: 13/446,618

(22) Filed: Apr. 13, 2012

(30) Foreign Application Priority Data
Jun. 29, 2010 (JP) 2010-147781

(51) Int. Cl. A61K 47/40; A23L 1/03

(52) U.S. Cl. 514/777; 426/658

(57) ABSTRACT

It has been necessary to use a relatively large amount of cyclodextrin in order to produce a composite material, but the amount of the cyclodextrin needs to be reduced from the viewpoint of costs; in this respect, the present invention provides a composite material comprising a food or active pharmaceutical ingredient, a phytosterol ester, a medium-chain fatty acid triacylglyceride, and a cyclodextrin.

### BACKGROUND OF THE INVENTION

1. Field of the Invention

The present invention relates to a composite material comprising a food or active pharmaceutical ingredient, and to a method for producing the composite material.

2. Description of the Related Art

Capsaicins, which are pungent components of capsicum pepper, are one example of the lipophilic components having an irritating taste or odor. Capsaicins are known to have various actions useful for organisms, such as an action of appetite stimulation, actions of vasodilation and vasoconstriction, an action of increasing salivation, an action of increasing gastric acid secretion, an action of increasing peristaltic movement of the intestinal tract, an action of decreasing the cholesterol level in the circulatory system, an action of increasing energy metabolism, and an action of increasing the release of bioactive peptides. However, capsaicins have a strong pungency, and hence the application range thereof to foods and beverages has been limited.

To inhibit the pungency of capsaicins, novel capsaicinoid glycosides have been proposed which are obtained by modifying the molecular structure of capsaicinoids to eliminate their strong pungency (Patent Document 1). However, the capsaicinoid glycosides are novel chemically synthesized compounds, and hence are not approved yet for use in foods or beverages.

Moreover, a masking agent which is characterized by including a polyglycerin condensed ricinoleate and a food including the masking agent have been proposed (Patent Document 2). Specifically, a masking agent-containing chili oil has been disclosed which is obtained by adding 0.1% of a capsicum pepper extract oil and 0.5% of hexaglycerin condensed ricinoleate to sesame oil. However, the application range of this masking agent is limited to foods and beverages containing a large amount of an oil component. In addition, this masking agent is likely to affect the flavor of foods and beverages because of a waxy odor thereof.

Furthermore, an edible microcapsule has been proposed which is characterized in that an edible fat and fatty oil containing capsaicin is used as a core material, a wall membrane is formed of a protein and a coacervate agent, and a transglutaminase is used as an agent for curing and crosslinking the wall membrane (Patent Document 3). However, the present inventors actually prepared this edible microcapsule and stirred this edible microcapsule with hot water (at 97° C.) in a beaker. As a result, the core material leaked, and floating oil was observed. Therefore, the edible microcapsule was found not to be suitable for beverages.

In addition to capsicum pepper, for example, turmeric has been conventionally used as a spice, a yellow pigment, and a crude drug. Recently, turmeric has attracted attention as a health food material. Turmeric has been commercially available as processed foods, processed beverages, tablets, and the like. A soft capsule preparation is proposed which is capable of masking the characteristic bitter taste of turmeric and of effectively preventing change of turmeric with time (Patent Document 4). The soft capsule preparation is characterized by comprising a capsule base member mainly containing gelatin, and a target content with which the capsule base member is filled, wherein the gelatin is prepared by being dissolved in electrolyzed-reduced water with a pH of 8 to 10 and with a redox potential of −100 mV to −200 mV.

As described above, the excellent and effective actions of naturally occurring materials typified by capsicum pepper and turmeric have attracted attention in the fields of foods and pharmaceuticals. With increase in demands for natural products and enhancement of health consciousness, people who want to be healthy favor natural and mild actions of not only traditional Chinese herbal medicines and crude drugs but also various materials, such as spices and herbs. Accordingly, these materials have been increasingly used as raw materials for health foods and pharmaceuticals.

Under such circumstances, the present applicant has filed a patent application for a composite material comprising: a lipophilic component having an irritating taste and/or odor; a phytosterol ester; and a cyclodextrin, as a material capable of effectively inhibiting an irritating taste and/or odor of a lipophilic component having an irritating taste and/or odor, which is typified by pungent components of capsaicins, and bitter components of a turmeric extract, and also for a method for producing the composite material (Patent Document 5).

In addition, the present applicant also has filed a patent application for a method for preventing decomposition/deterioration of a lipophilic component in the presence of water (PCT/JP2009/71473). This method comprises: forming a composite material containing a lipophilic component, a phytosterol ester, and a cyclodextrin; and storing the lipophilic component in the form of the composite material in the presence of water.

Moreover, the present applicant has filed a patent application for a composite material comprising a phytosterol ester, a hydrophilic component surface-treated with a surfactant, and a cyclodextrin, in order to provide a material which comprises a hydrophilic component having an irritating taste or odor such as a bitter or pungent taste, and which is capable of effectively inhibiting the irritating taste or odor of the hydrophilic component, and to provide a method for producing the material, as well as in order to provide a material which comprises a hydrophilic component decomposable in the presence of water, and which is capable of effectively preventing the decomposition of the hydrophilic component with the elapse of time, and to provide a method for producing the material (Japanese Patent Application No. 2008-328263).
Patent Document 1: Japanese Patent No. 3156240
Patent Document 2: Japanese Patent Application Publication No. 2002-65177
Patent Document 3: Japanese Patent Application Publication No. 2003-47432
Patent Document 4: Japanese Patent No. 4469660
Patent Document 5: International Publication No. WO2009/005005

### SUMMARY OF THE INVENTION

When the composite material is manufactured and used as a business, the present applicant has encountered a problem of the high costs of raw materials, particularly of the cyclodextrin. To solve this problem, it is necessary to reduce the amount of the cyclodextrin, while a desired effect is obtained.

The present invention provides a composite material comprising a food or active pharmaceutical ingredient; a phytosterol ester; a medium-chain fatty acid triacylglyceride; and a cyclodextrin.

The present invention also provides a composition comprising the composite material.

Moreover, the present invention provides a liquid composition comprising: the composite material; water; and a thickener, wherein the composite material is in a water-dispersed form.

Further, the present invention provides a method for producing a composite material comprising a food or active pharmaceutical ingredient, a phytosterol ester, a medium-chain fatty acid triacylglyceride, and a cyclodextrin, the method comprising: dissolving the food or active pharmaceutical ingredient in the phytosterol ester and the medium-chain fatty acid triacylglyceride; preparing a mixture containing the cyclodextrin and water; and mixing the phytosterol ester and the medium-chain fatty acid triacylglyceride, in which the food or active pharmaceutical ingredient is dissolved, with the mixture, to form the composite material.

### EFFECTS OF THE INVENTION

The present invention makes it possible to provide a composite material comprising: a food or active pharmaceutical ingredient; a phytosterol ester; a medium-chain fatty acid triacylglyceride, and a cyclodextrin, with the amount of the cyclodextrin being reduced.

### BRIEF DESCRIPTION OF THE DRAWINGS

FIG. 1 is a graph showing change in allyl amount in Reference Example 1 and Reference Comparative Example 1.

FIG. 2 is a graph showing change in capsaicin amount in Reference Example 2 and Reference Comparative Example 2.

FIG. 3 is a graph showing change in ratio of remaining capsinoids in Reference Example 3 and Reference Comparative Examples 3-1 and 3-2.

FIG. 4 is a graph showing change of gingerol during storage in Reference Example 4 and Reference Comparative Example 4.

FIG. 5 is a graph showing change of shogaol during storage in Reference Example 4 and Reference Comparative Example 4.

FIG. 6 is a graph showing change of piperine during storage in Reference Example 5 and Reference Comparative Example 5.

### DESCRIPTION OF THE PREFERRED EMBODIMENTS

A composite material of the present invention comprises: a food or active pharmaceutical ingredient; a phytosterol ester; a medium-chain fatty acid triacylglyceride; and a cyclodextrin.

The food or active pharmaceutical ingredient contained in the composite material of the present invention is not particularly limited, and the present invention is particularly effective when directed to a raw material having an irritating taste and/or odor, or a raw material which easily degrades during storage and is unstable. The composite material of the present invention presumably has a structure in which the food or active pharmaceutical ingredient is incorporated into a lipid comprising the phytosterol ester and the medium-chain fatty acid triacylglyceride, and is shut off from the outside, i.e., has a kind of a capsule structure. Accordingly, the food or active pharmaceutical ingredient employed in the composite material of the present invention is not particularly limited, as long as the food or active pharmaceutical ingredient can be incorporated into the lipid comprising the phytosterol ester and the medium-chain fatty acid triacylglyceride.

Accordingly, when the food or active pharmaceutical ingredient is a lipophilic component, the food or active pharmaceutical ingredient has a high affinity for the phytosterol ester and the medium-chain fatty acid triacylglyceride. Hence, in such a case, the composite material can be formed by mixing the food or active pharmaceutical ingredient directly with the phytosterol ester, the medium-chain fatty acid triacylglyceride, and the cyclodextrin. Examples of the lipophilic component include capsaicins, which fall into a class of the lipophilic pungent components. The capsaicins include capsaicin, dihydrocapsaicin, nordihydrocapsaicin, homocapsaicin, vanillylnonanamide, and vanillyl butyl ether. Capsicum pepper extracts such as capsicum oleoresins can be suitably used as a raw material containing capsaicins, because the extracts contain a large amount of capsaicins.

Meanwhile, examples of the lipophilic component other than the capsaicins include (6)-gingerol, (6)-shogaol, zingerone, and (8),(10)-shogaol, which are pungent components of ginger; piperine and piperanine, which are pungent components of pepper; sanshool, which is a pungent component of Japanese pepper; and the like. A pepper extract, a ginger extract, and a Japanese pepper extract can be suitably used as raw materials containing pungent components of ginger, pepper, and Japanese pepper, respectively.

In addition to the pungent components, the present invention can be applied to lipophilic bitter components such as a turmeric extract containing a lipophilic component having a bitter taste. Moreover, the present invention can be applied not only to the above-described lipophilic components of spices, but also to unsaturated fatty acids such as docosahexaenoic acid and eicosapentaenoic acid.

In addition, the composite material of the present invention is found to be capable of preventing the decomposition of the lipophilic component due to, for example, interaction with water, or interaction with light, an enzyme, oxygen, heat, or the like in the presence of water. In other words, the composite material of the present invention stabilizes the lipophilic component, and improves the storability thereof. Accordingly, for example, substances having a structure analogous to those of capsaicins but having no pungency, such as capsinoids, unsaturated fatty acids, curcumin, which is a pigment component of turmeric, and the like can be suitably used as the lipophilic component. The composite material of the present invention is effective for improvement in stability of these substances.

When the food or active pharmaceutical ingredient is a hydrophilic component, the food or active pharmaceutical ingredient is preferably a hydrophilic component surface-treated with a surfactant in order to increase the affinity for the phytosterol ester and the medium-chain fatty acid triacylglyceride. Examples of the hydrophilic components include caffeine, B vitamins, betanin, isobetanin, and the like.

Caffeine is a component contained in coffee, black tea, and the like. Caffeine has a strong bitter taste, and is known to have physiological effects such as sleepiness prevention, stress relaxation, and obesity prevention.

B vitamins collectively refer to eight kinds of water-soluble vitamins, including vitamin B1, vitamin B2, niacin, pantothenic acid, vitamin B6, vitamin B12, folic acid, and biotin, and are also referred to as a vitamin B complex. B vitamins are often contained in beans or seeds such as soybean, pork or beef liver, and the like. B vitamins are used in organisms as raw materials of coenzymes, and hence are necessary for metabolism in the body.

Betanin and isobetanin are main components of the red pigment contained in red beet, and used as a natural edible pigment. The pigment has a vivid red color, and undergoes less change in color due to pH. The pigment is known to be stable in the pH range of 4 to 7, but is unstable to heat.

The phytosterol ester used in the present invention is a substance obtained by ester-bonding a fatty acid to a hydroxyl group in the sterol skeleton of a phytosterol. Examples of a method for producing the phytosterol ester include an enzymatic method utilizing an enzyme, and the like. Examples of the enzymatic method include a method of obtaining the phytosterol ester by mixing a phytosterol and a fatty acid and causing a reaction therebetween (at 30 to 50° C. for approximately 48 hours) with a lipase or the like used as a catalyst; and the like. Examples of other synthesis methods include a method of obtaining a phytosterol ester by esterification of a phytosterol produced from soybean or the like with a fatty acid obtained from rapeseed oil, corn oil, or her like, by dehydration in the presence of a catalyst; and the like.

Examples of the phytosterol include sterols contained in vegetable fats and fatty oils, and the like. For example, the phytosterol may be one extracted and purified from a vegetable fat or fatty oil of soybean, rapeseed, cottonseed, or the like. The phytosterol may be a mixture containing β-sitosterol, campesterol, stigmasterol, brassicasterol, fucosterol, dimethylsterol, and the like. For example, a soybean sterol contains 53 to 56% of sitosterol, 20 to 23% of campesterol, and 17 to 21% of stigmasterol. A phytosterol which is commercially available as “Phytosterol F” (produced by TAMA BIOCHEMICAL CO., LTD.) can also be used as the phytosterol.

The fatty acid may be plant derived, for example, derived from rapeseed oil or palm oil, or may be animal derived. Examples of the fatty acid include myristic acid, stearic acid, palmitic acid, arachidonic acid, oleic acid, linoleic acid, α-linolenic acid, γ-linolenic acid, eicosapentaenoic acid, docosahexaenoic acid, palmitoleic acid, lauric acid, and the like.

Preferred examples of the phytosterol ester include phytosterol esters each obtained from a phytosterol derived from soybean and a fatty acid derived from rapeseed oil; phytosterol esters each obtained from a phytosterol derived from soybean or rapeseed and a fatty acid derived from palm oil; and the like. The former include “San Sterol NO. 3” of San-Ei Gen F. F. I., Inc., and the like. The latter include “Phytosterol Fatty Acid Ester” of TAMA BIOCHEMICAL CO., LTD., and the like.

The medium-chain fatty acid triacylglyceride used in the present invention is a triglyceride whose constituent is a medium-chain fatty acid, namely octanoic acid (trivial name: caprylic acid), which is a fatty acid having 8 carbon atoms, or decanoic acid (trivial name: capric acid), which is a fatty acid having 10 carbon atoms.

The cyclodextrin used in the present invention is a cyclic non-reducing maltooligosaccharide, whose constitutional unit is glucose. Any of α-cyclodextrin with six glucose units, β-cyclodextrin with seven glucose units, and γ-cyclodextrin with eight glucose units can be used as the cyclodextrin. γ-Cyclodextrin is preferable from the viewpoint that γ-cyclodextrin is decomposed by human digestive enzymes, and has high solubility in water, and accordingly γ-cyclodextrin is used for foods and beverages, particularly for beverages, without reluctance.

The composite material of the present invention is obtainable by mixing the food or active pharmaceutical ingredient, the phytosterol ester, the medium-chain fatty acid triacylglyceride, and the cyclodextrin with each other in the presence of water. For producing the composite material of the present invention, the amounts of the phytosterol ester and the medium-chain fatty acid triacylglyceride are, for example, preferably such that the total amount of the phytosterol ester and the medium-chain fatty acid triacylglyceride is 0.5 to 30000 parts by weight with respect to 1 part by weight of the food or active pharmaceutical ingredient, although the amounts vary depending on the target food or active pharmaceutical ingredient. Meanwhile, the amount of the cyclodextrin is, for example, preferably 0.00135 to 135 parts by weight with respect to 1 part by weight of the total amount of the phytosterol ester and the medium-chain fatty acid triacylglyceride. When a homogenizing process is conducted with a homogenizer, the amount of the cyclodextrin is more preferably 0.00135 to 15 parts by weight with respect to 1 part by weight of the total amount of the phytosterol ester and the medium-chain fatty acid triacylglyceride. In addition, the amount of water coexistent during the production of the composite material is, for example, preferably 0.01 to 100 parts by weight, and more preferably 0.1 to 10 parts by weight, with respect to 1 part by weight of the cyclodextrin. In addition, when the surface of the hydrophilic component is treated with a surfactant, the amount of the surfactant is, for example, preferably 0.0001 to 10 parts by weight, and more preferably 0.0001 to 10 parts by weight, with respect to 1 part by weight of the hydrophilic component. In addition, when the composite material of the present invention is produced, the mixing is preferably conducted under heating at 40 to 90° C., and more preferably 50 to 85° C.

In addition, in the composite material of the present invention, the ratio between the phytosterol ester and the medium-chain fatty acid triacylglyceride is 9:1 to 1:9, and more preferably 7:3 to 3:7 in terms of weight ratio.

More specifically, the composite material of the present invention can be produced by any one of the following methods (1) to (3); however, the method (1) is particularly preferable in order to more effectively inhibit the taste or odor of the food or active pharmaceutical ingredient:

(1) a method comprising dissolving the food or active pharmaceutical ingredient in a phytosterol ester and the medium-chain fatty acid triacylglyceride, preparing a mixture containing the cyclodextrin and water, and mixing the phytosterol ester and the medium-chain fatty acid triacylglyceride, in which the food or active pharmaceutical ingredient is dissolved, with the mixture;

(2) a method comprising preparing a mixture containing the cyclodextrin, water, the phytosterol ester, and the medium-chain fatty acid triacylglyceride, and mixing the food or active pharmaceutical ingredient and water with the mixture; and

(3) a method comprising preparing a mixture containing the food or active pharmaceutical ingredient and the cyclodextrin, and mixing water, the phytosterol ester, and the medium-chain fatty acid triacylglyceride with the mixture.

Of these methods, the method (1) is more specifically described below. Specifically, when the food or active pharmaceutical ingredient is, for example, capsaicins, 1 part by weight of the capsaicins are dissolved in 30 to 30000 parts by weight, in total, of the phytosterol ester and the medium-chain fatty acid triacylglyceride in the step of dissolving the food or active pharmaceutical ingredient in the phytosterol ester and the medium-chain fatty acid triacylglyceride, although this ratio varies depending on the target food or active pharmaceutical ingredient. For dissolving the food or active pharmaceutical ingredient in the phytosterol ester, the food or active pharmaceutical ingredient is preferably dissolved in such a way that the food or active pharmaceutical ingredient is added to the phytosterol ester and the medium-chain fatty acid triacylglyceride, and the resultant mixture is warmed to 40 to 80° C., preferably 50 to 70° C. Alternatively, the food or active pharmaceutical ingredient may be dissolved in such a way that the phytosterol ester and the medium-chain fatty acid triacylglyceride are warmed to 40 to 80° C., preferably 50 to 70° C. in advance, and then the food or active pharmaceutical ingredient is added thereto.

In the step of preparing the mixture containing the cyclodextrin and water, the amounts of the cyclodextrin and water are not particularly limited, as long as the composite material can be formed later with these amounts. For example, the amount of the cyclodextrin is, for example, 0.00135 to 135 parts by weight with respect to 1 part by weight of the total amount of the phytosterol ester and the medium-chain fatty acid triacylglyceride. When a homogenizing process is conducted with a homogenizer, the amount of the cyclodextrin is preferably 0.00135 to 15 parts by weight with respect to 1 part by weight of the total amount of the phytosterol ester and the medium-chain fatty acid triacylglyceride. Meanwhile, the amount of water is, for example, 0.01 to 100 parts by weight, and preferably 0.1 to 10 parts by weight, with respect to 1 part by weight of the cyclodextrin.

In the step of mixing the phytosterol ester, in which the food or active pharmaceutical ingredient is dissolved, with the mixture, the mixing is continued until the composite material of the present invention is formed. The thus formed composite material is deposited as particulate matters in the lower part of water, after the mixing is stopped and the mixture is allowed to stand for a while. Note that the mixing here is performed preferably by using a mixing apparatus with high shearing force, such as a kneader, for forming the composite material by thoroughly kneading these components.

The obtained composite material can be in any form. For example, the composite material may be formed into a powder form or a granular form by using an excipient or the like. In addition, the composite material may be in a liquid form or a paste form, where the composite material is dispersed or emulsified in a solvent such as water.

The thus obtained composite material of the present invention is advantageous in that the irritating taste and/or odor of the lipophilic component is effectively inhibited. The inhibition of the irritating taste and/or odor of the composite material of the present invention has a mechanism different form that of what is called as masking involving addition of, for example, a sweet component or the like. It is uncertain what structure the composite material of the present invention has. However, the lipophilic component contained in the composite material of the present invention is presumably at least in such a state that the lipophilic component cannot bind to the receptor of the taste.

In addition, the composite material of the present invention is advantageous in that no separation of an oil component occurs. Incidentally, when a composite material is produced by using a different oil instead of the phytosterol ester, a homogeneous composite material cannot be obtained, and separation of an oil component occurs. Hence, when blended into a beverage or the like, the composite material has a problem that the oil component floats and attaches to the inner surface of a container. In contrast, the composite material of the present invention is advantageous in that the use of the phytosterol ester makes it possible to obtain a homogeneous composite material, and that an oil component does not separate, and hence no oil component attaches onto the inner surface of a container, when the composite material is blended into a beverage or the like.

Moreover, the composite material of the present invention is also thermally stable. For example, even when the composite material is blended into a food or beverage, and heated to 65 to 100° C., the irritating taste and/or odor of the lipophilic component can be inhibited, and no separation of an oil component occurs.

The composite material of the present invention is easily dispersed in water. Hence, the composite material can be blended into foods, beverages, pharmaceutical drugs, cosmetics, and the like, and provided as various compositions. More specifically, examples of foods and beverages into which the composite material of the present invention is blended include beverages, jellies, tablets, and the like. Here, a case where the composite material of the present invention is blended into a beverage is taken as an example. A heat-sterilized packed beverage can be produced, for example, as follows. Specifically, the composite material of the present invention is added to water. An acidulant is added thereto to adjust the pH to 4.0 or less, and preferably 2.5 to 3.5. Raw materials such as a sweetener, a fruit juice, a flavor, a coloring matter, and vitamin C are added thereto, and mixed. This mixture is heated to 65 to 100° C. for sterilization treatment, and then packed and sealed in a container. Moreover, a packed jelly can be produced by adding a gelatinizing agent to the raw materials.

The composition of the present invention can also be provided as a liquid composition which comprises the composite material, water, and a thickener, and which is in a form where the composite material is dispersed in water. Specifically, although the composite material tends to be deposited in water, the inclusion of the thickener makes it possible to provide a liquid composition in which the composite material is dispersedly held in water. In addition, this liquid composition can also be provided as a packed liquid composition such as a packed beverage. In this case, there are advantages in that no separation of an oil component occurs in the container, and hence no oil component attaches onto the inner surface of the container.

Here, examples of the thickener include gellan gum, fermentation-derived cellulose, xanthan gum, gum arabic, tamarind gum, guar gum, locust bean gum, karaya gum, tara gum, agar, gelatin, pectin, soybean polysaccharide, CMC (carboxymethyl cellulose), carrageenan, microcrystalline cellulose, propylene glycol alginate, and the like. Of these, fermentation-derived cellulose is preferably used, from the viewpoint that the composite material has a favorable texture, when dispersed uniformly in water and ingested orally.

The amount of the thickener is not particularly limited, as long as the composite material can be dispersed in water with the amount. For example, the liquid composition preferably contains 0.01 to 1.0% by weight of the thickener.

### EXAMPLES

### Example 1

### (1) Production of Composite Material

With string, 0.00284 parts by weight of a capsicum oleoresin (content of capsaicins: 35.21% by weight), 0.0303 parts by weight of a medium-chain fatty acid triacylglyceride (“MCT-1” manufactured by J-OIL MILLS, Inc.), and 0.2697 parts by weight of a phytosterol ester (“San Sterol No. 3” manufactured by San-Ei Gen F. F. I., Inc.) were heated to 80° C.

Separately, 1.1 parts by weight of γ-cyclodextrin was dissolved in 1.097 parts by weight of water (80° C.).

To the γ-cyclodextrin solution, the solution of the capsicum oleoresin, the medium-chain fatty acid triacylglyceride, and the phytosterol ester was added. After a preliminary homogenization treatment (at 5000 rpm for 10 minutes), a high-pressure homogenization treatment (at 100 MPa, a three-plunger type) was further conducted. Thus, a composite material was obtained.

### (2) Production of Beverage

Citric acid, trisodium citrate, xanthan gum, sucralose, and water were added to the composite material, and the substances were mixed with each other with stirring. Subsequently, the mixture was heated to 93° C. with string, and heat sterilized by being held for 3 minutes. After that, the mixture was packed into a container, and cooled. Thus, a packed beverage was produced. The thus obtained beverage had a weaker pungency than the beverage obtained in the following Comparative Example 1. Note that the blending ratio of the beverage is as shown in the following Table 1.

### Comparative Example 1

### (1) Production of Composite Material

With string, 0.00284 parts by weight of a capsicum oleoresin (content of capsaicins: 35.21% by weight) and 0.3000 parts by weight of a phytosterol ester (“San Sterol No. 3” manufactured by San-Ei Gen F. F. I., Inc.) were heated to 80° C.

Separately, 1.1 parts by weight of γ-cyclodextrin was dissolved in 1.097 parts by weight of water (80° C.).

To the γ-cyclodextrin solution, the solution of the capsicum oleoresin and the phytosterol ester was added. After a preliminary homogenization treatment (at 5000 rpm for 10 minutes), a high-pressure homogenization treatment (at 100 MPa, a three-plunger type) was further conducted. Thus, a composite material was obtained.

### (2) Production of Beverage

Citric acid, trisodium citrate, xanthan gum, sucralose, and water were added to the composite material, and the substances were mixed with each other with stirring. Subsequently, the mixture was heated to 93° C. with string, and heat sterilized by being held for 3 minutes. After that, the mixture was packed into a container, and cooled. Thus, a packed beverage was produced. A strong pungency was noticed from the thus obtained beverage. Note that the blending ratio of the beverage is as shown in the following Table 1.

TABLE 1

| | | Comparative |
|---|---|---|
| Raw material (parts by weight) | Example 1 | Example 1 |
| Capsicum oleoresin | 0.00284 | 0.00284 |
| (capsaicins: 35.21% by weight) | | |
| Medium-chain fatty acid triacylglyceride | 0.0303 | — |
| (“MCT-1” manufactured | | |
| by J-OIL MILLS, Inc.) | | |
| Phytosterol ester | 0.2697 | 0.3000 |
| (“San Sterol No. 3” manufactured | | |
| by San-Ei Gen F. F. I., Inc.) | | |
| γ-Cyclodextrin | 1.1 | 1.1 |
| (“CAVAMAX-W8 Food” manufactured | | |
| by Cyclochem Co., Ltd.) | | |
| Water | 1.097 | 1.097 |
| Citric acid | 0.50 | 0.50 |
| Trisodium citrate | 0.25 | 0.25 |
| Sucralose | 0.017 | 0.017 |
| Xanthan gum | 0.05 | 0.05 |
| (“San Ace” manufactured by San-Ei | | |
| Gen F. F. I., Inc.) | | |
| water | 96.68316 | 96.68316 |
| Total | 100 | 100 |
| Pungency evaluation | Weak | Strong |
| | pungency | pungency was |
| | was noticed | noticed |

### Examples 2 and 3

Composite materials and beverages were produced in the same manner as in Example 1, except that the ratio between the phytosterol ester and the medium-chain fatty acid triacylglyceride was changed to the ratios shown in Table 2. Table 2 shows sensory evaluation results of the beverages.

### Comparative Example 2

With string, 0.00284 parts by weight of a capsicum oleoresin (content of capsaicins: 35.21% by weight) and 0.3000 parts by weight of a medium-chain fatty acid triacylglyceride (“MCT-1” manufactured by J-OIL MILLS, Inc.) were heated to 80° C.

Separately, 1.1 parts by weight of γ-cyclodextrin was dissolved in 1.097 parts by weight of water (80° C.).

To the γ-cyclodextrin solution, the solution of the capsicum oleoresin and the phytosterol ester was added. A preliminary homogenization treatment (at 5000 rpm for 10 minutes) was conducted. However, the fluidity was lost at this point, and the high-pressure homogenizer was unable to be conducted.

TABLE 2

| | | | | Comp. | Comp. |
|---|---|---|---|---|---|
| | Ex. 1 | Ex. 2 | Ex. 3 | Ex 1 | Ex 2 |
| Ratio | 9:1 | 6:4 | 3:7 | 10:0 | 0:10 |
| (phytosterol | | | | | |
| ester:medium- | | | | | |
| chain fatty acid | | | | | |
| triacylglyceride) | | | | | |
| Pungency | Weak | Almost | Almost | Strong | (Evaluation |
| evaluation | pungency | no | no | pun- | was |
| | was | pun- | pun- | gency | impossible) |
| | noticed | gency | gency | was | |
| | | was | was | noticed | |
| | | noticed | noticed | | |

Note that approximately 8150 parts by weight of γ-cyclodextrin was used, in Example 1 of Patent Document 5, with respect to 1 part by weight of capsaicins to obtain a beverage from which almost no pungency was noticed. In contrast, it was found that, in Example 2 and Example 3 of the present invention, the use of only approximately 1100 parts by weight (approximately 1/7.4) of γ-cyclodextrin with respect to 1 part by weight of capsaicins astonishingly made it possible to obtain beverages from which almost no pungency was noticed, as in the case with Example 1 of Patent Document 5.

### Example 4

### (1) Production of Composite Material

To 0.06 parts by weight of a phytosterol ester (“San Sterol No. 3” manufactured by San-Ei Gen F. F. I., Inc.) and 0.04 parts by weight of a medium-chain fatty acid triglyceride (“MCT-1” manufactured by J-OIL MILLS, Inc.), which were heated to 80° C., 0.015 parts by weight of a ginger extract (shogaol: 10%) was added, and dissolved therein.

Separately, 0.37 parts by weight of γ-cyclodextrin and 0.37 parts by weight of water were mixed with each other by using a TK homomixer, while being heated to 80° C.

To the γ-cyclodextrin solution, 0.115 parts by weight of the oil phase in which the ginger extract was dissolved was added. While continuously being heated to 80° C., the mixture was stirred with a TK homomixer to conduct preliminary emulsification.

After the preliminary emulsification, the mixture was passed through a high-pressure homogenizer (LAB1000 manufactured by SMT Co., Ltd., pressure: 100 MPa). Thus, a ginger extract-containing composite material was prepared.

### (2) Production of Beverage

Into 97.705 parts by weight of water, 0.855 parts by weight of the composite material, 0.3 parts by weight of citric acid, 0.12 parts by weight of trisodium citrate, 0.72 parts by weight of γ-cyclodextrin, and 0.3 parts by weight of a phytosterol preparation (manufactured by San-Ei Gen) were dispersed, and the dispersion was stirred with a mixer for 30 seconds. Thus, a model beverage containing the ginger extract composite material was prepared. The model beverage was heated up to 93° C., sterilized by being held at 90° C. for 3 minutes, and then packed into a pouch. Thereafter, the pouch was held in a constant-temperature water bath at 83° C. for 5 minutes to perform a second sterilization.

### Comparative Example 3

### (1) Production of Composite Material

To 0.06 parts by weight of a phytosterol ester (“San Sterol No. 3” manufactured by San-Ei Gen F. F. I., Inc.), which was heated to 80° C., 0.015 parts by weight of a ginger extract (shogaol: 10%) was added, and dissolved therein.

Separately, 0.37 parts by weight of γ-cyclodextrin and 0.37 parts by weight of water were mixed with each other by using a TK homomixer, while being heated to 80° C.

To the γ-cyclodextrin solution, 0.075 parts by weight of the oil phase in which the ginger extract was dissolved was added. While continuously being heated to 80° C., the mixture was stirred with a TK homomixer to conduct preliminary emulsification.

After the preliminary emulsification, the mixture was passed through a high-pressure homogenizer (LAB1000 manufactured by SMT Co., Ltd., pressure: 100 MPa). Thus, a ginger extract-containing composite material was prepared.

### (2) Production of Beverage

Into 97.745 parts by weight of water, 0.815 parts by weight of the composite material, 0.3 parts by weight of citric acid, 0.12 parts by weight of trisodium citrate, 0.72 parts by weight of γ-cyclodextrin, and 0.3 parts by weight of a phytosterol preparation (manufactured by San-Ei Gen) were dispersed, and the dispersion was stirred with a mixer for 30 seconds. Thus, a model beverage containing a ginger extract composite material was prepared. The model beverage was heated up to 93° C., sterilized by being held at 90° C. for 3 minutes, and then packed into a pouch. Thereafter, the pouch was held in a constant-temperature water bath at 83° C. for 5 minutes to conduct a second sterilization.

TABLE 3

| | | Comparative |
|---|---|---|
| Raw material (parts by weight) | Example 4 | Example 3 |
| Ginger extract (shogaol: 10%) | 0.015 | 0.015 |
| Phytosterol ester | 0.06 | 0.06 |
| (“San Sterol No. 3” manufactured by San-Ei | | |
| Gen F. F. I., Inc.) | | |
| Medium-chain fatty acid triglyceride | 0.04 | — |
| (“MCT-1” manufactured by | | |
| J-OIL MILLS, Inc.) | | |
| γ-Cyclodextrin | 0.37 | 0.37 |
| Water | 0.37 | 0.37 |
| Citric acid | 0.3 | 0.3 |
| Trisodium citrate | 0.12 | 0.12 |
| γ-Cyclodextrin | 0.72 | 0.72 |
| Phytosterol preparation | 0.3 | 0.3 |
| (“San Sterol No. 1” manufactured by San-Ei | | |
| Gen F. F. I., Inc.) | | |
| Water | 97.705 | 97.745 |
| Total | 100 | 100 |

### (Stability Evaluation)

The model beverages prepared in Example 4 and Comparative Example 3 were stored at 60° C. The amount of shogaol in each sample before the storage and after two-week storage was quantitatively determined by liquid chromatography. As the ratio of remaining shogaol, a value of each sample determined after the two-week storage was represented by percentage, with a value of each sample before the storage (0 weeks) being employed as 100%. Table 4 shows the results.

### Pretreatment Method for Liquid Chromatography

Each model beverage (25 g) was centrifuged (at 3000 rpm for 10 minutes), and then the supernatant was removed. To the deposit, 3 ml of DMSO (dimethyl sulfoxide) was added, and the mixture was ultrasonicated to dissolve the deposit. Moreover, the mixture was diluted with methanol to 50 ml, filtered through a 0.45-μm filter, and then used as a test liquid.

### Measurement Conditions for Liquid Chromatography

### UV: 282 nm

### Column: ODS C18

Flow rate: 1.0 ml/min
Injection amount: 20 μl
Analysis time: 30 minutes
Mobile phase: Acetonitrile:Water:THF (tetrahydrofuran)=45:50:5

TABLE 4

| | | Comparative |
|---|---|---|
| | Example 4 | Example 3 |
| Ratio of remaining shogaol (%) | 97.4 | 90.3 |

As is apparent from Table 4, the decomposition of shogaol was more prevented in Example 4 than in Comparative Example 3. In other words, it has been found that the addition of the medium-chain fatty acid triglyceride enables further improvement of the stability of the active ingredients in the ginger extract.

Hereinafter, Examples of PCT/JP2009/71473 are described as Reference Examples of the food or active pharmaceutical ingredient (lipophilic component) to which the present invention can be applied.

### Reference Example 1

To 5.67 parts by weight of a phytosterol ester melted by heating to 60° C., 0.63 parts by weight of a mustard essential oil was added, and dissolved therein. Meanwhile, 62.4 parts by weight of γ-cyclodextrin and 31.3 parts by weight of water (75° C.) were added to a mortar, and mixed with each other by using a pestle to obtain a paste. To this paste, the above-described phytosterol ester in which the mustard essential oil was dissolved was added, and the mixture was kneaded in a hot water (75° C.) for 10 minutes. After completion of the kneading, water in an amount equivalent to water lost due to vaporization was added thereto, and the mixture was kneaded again to homogeneity. The blended amounts (g) in Reference Example 1 are shown in the following Table 5.

### Reference Comparative Example 1

Into a mortar, 66.24 parts by weight of γ-cyclodextrin and 33.13 parts by weight of water (60° C.) were added, and mixed with each other by using a pestle to obtain a paste. To this paste, 0.63 parts by weight of a mustard essential oil was added, and the mixture was kneaded in hot water (75° C.) for 10 minutes. After completion of the kneading, water in an amount equivalent to water lost due to vaporization was added thereto, and the mixture was kneaded again to homogeneity. The blended amounts (g) in Reference Comparative Example 1 are shown in the following Table 5.

TABLE 5

| | | Reference |
|---|---|---|
| | Reference | Comparative |
| Raw material | Example 1 | Example 1 |
| Mustard essential oil | 0.63 g | 0.63 g |
| (allyl isothiocyanate content: 97% by weight) | | |
| Phytosterol ester | 5.67 g | — |
| (San Sterol No. 3 manufactured by San-Ei | | |
| Gen F. F. I., Inc.) | | |
| γ-Cyclodextrin | 62.40 g | 66.24 g |
| Water | 31.30 g | 33.13 g |
| Total | 100.00 g | 100.00 g |

### (Storage Method)

To 1 part by weight of each of the samples obtained in Reference Example 1 and Reference Comparative Example 1, 5 parts by weight of water was added, and the sample was uniformly dispersed therein. GC vials were filled up with the samples of the composite materials dispersed in water, and then each GC vial was tightly closed with a cap, and sealed in an aluminum pouch. These vials were stored at 50° C.

### (GC Measurement)

The samples stored for 0 days (at the beginning of the storage), 1 day, and 6 days were diluted 100-fold with hexane, allowed to stand at room temperature for 16 to 18 hours, and filtered through a 0.45-μm filter to prepare GC samples. For the GC measurement, a FID detector was used. The GC measurement was carried out under the following conditions.

### Column: DB-WAX (Inner Diameter: 0.53 mm, Length: 30 m, Film Thickness: 1 μm)

Carrier gas: helium gas
Back pressure: 20 kPa
Injection temperature: 200° C.
Detector temperature: 220° C.
Temperature rise conditions: Temperature was raised from 100° C. to 180° C. (at a rate of temperature rise of 20° C./minute)

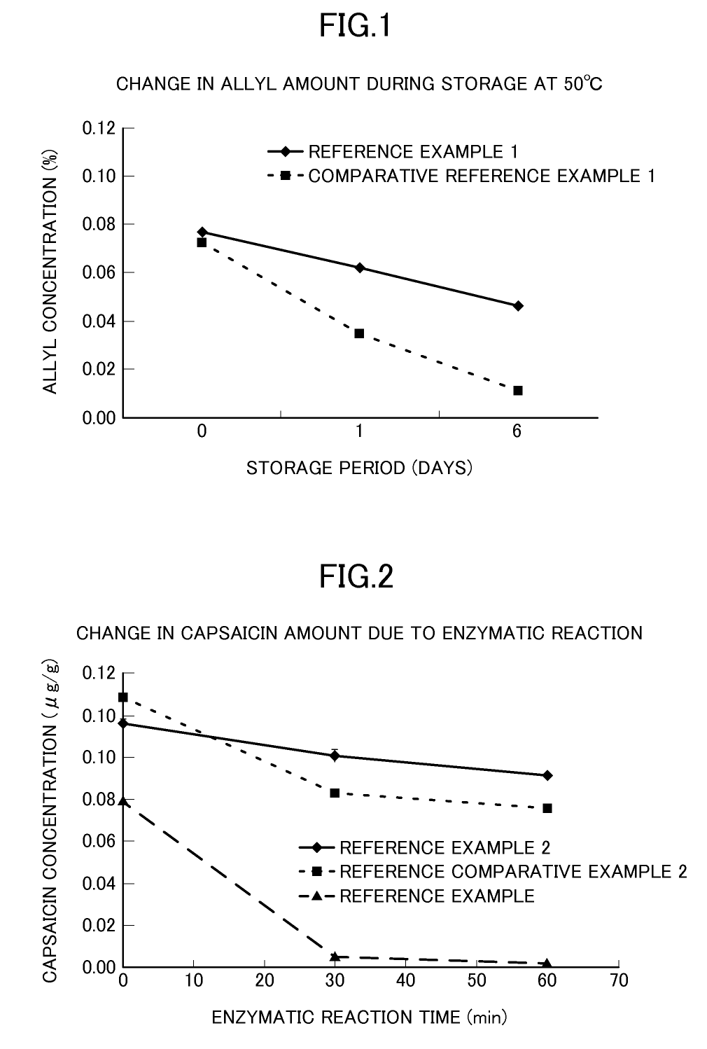

FIG. 1 shows change in allyl concentration. As shown in FIG. 1, the decomposition of allyl isothiocyanate in the oil was apparently prevented by storing the mustard essential oil in the presence of water in the form of the composite material which was formed together with the phytosterol ester and the γ-cyclodextrin. Note that the ratio of allyl isothiocyanate remaining after 6-day storage was 60.2% in Reference Example 1 and 15.5% in Reference Comparative Example 1, relative to that at the beginning of the storage.

### Reference Example 2

To 3.5 parts by weight of a phytosterol ester melted by heating to 60° C., 0.07 parts by weight of a capsicum oleoresin was added, and dissolved therein. Into a mortar, 64.3 parts by weight of γ-cyclodextrin and 32.13 parts by weight of water (60° C.) were added, and mixed with each other by using a pestle to obtain a paste. To this paste, the above-described phytosterol ester in which the capsicum oleoresin was dissolved was added, and the mixture was kneaded in hot water (60° C.) for 10 minutes. After completion of the kneading, water in an amount equivalent to water lost due to vaporization was added thereto, and the mixture was kneaded again to homogeneity. The blended amounts (g) in Reference Example 2 are shown in the following Table 6.

### Reference Comparative Example 2

Into a mortar, 66.6 parts by weight of γ-cyclodextrin and 33.33 parts by weight of water (60° C.) were added, and mixed with each other by using a pestle to obtain a paste. To this paste, 0.07 parts by weight of a capsicum oleoresin was added, and the mixture was kneaded in hot water (60° C.) for 10 minutes. After completion of the kneading, water in an amount equivalent to water lost due to vaporization was added thereto, and the mixture was kneaded again to homogeneity. The blended amounts (g) in Reference Comparative Example 2 are shown in the following Table 6.

TABLE 6

| | | Reference |
|---|---|---|
| | Reference | Comparative |
| Raw material | Example 2 | Example 2 |
| Capsicum oleoresin | 0.07 g | 0.07 g |
| (Content of capsaicins: 40% by weight) | | |
| Phytosterol ester | 3.50 g | — |
| (San Sterol No. 3 manufactured by San-Ei | | |
| Gen F. F. I., Inc.) | | |
| γ-Cyclodextrin | 64.30 g | 66.60 g |
| Water | 32.13 g | 33.33 g |
| Total | 100.00 g | 100.00 g |

### (Enzyme Addition and Storage Method)

Each of the samples obtained in Reference Example 2 and Reference Comparative Example 2 was diluted 10-fold with a 50 mM tris buffer (capsaicin concentration: 0.0028%). To this liquid, an acylase was added at a concentration of 0.05 u/ml. The mixture was shaken in a constant-temperature water bath at 37° C. to allow the reaction of the enzyme to proceed.

Meanwhile, as Reference Example, a capsaicin reagent (capsaicin content: 95% or more) manufactured by SIGMA was diluted with a 50 mM tris buffer such that the capsaicin concentration was the same as that (0.0028%) in Reference Example 2 and Reference Comparative Example 2. To this liquid, an acylase was added at a concentration of 0.05 u/ml. The mixture was shaken in a constant-temperature water bath at 37° C. in the same manner as in Reference Example 2 and Reference Comparative Example 2, to allow the reaction of the enzyme to proceed.

### (HPLC Measurement)

To 2 ml of each sample in which the reaction of the enzyme was allowed to proceed for 0 (at the beginning of the shaking) minutes, 30 minutes, or 60 minutes, 3 ml of water was added, and thus the volume thereof was adjusted to 5 ml. Moreover, 1 ml of 2.5 N NaOH was added thereto, followed by heating in boiling water at 100° C. for 10 minutes. After the heating, 20 ml of methanol was added thereto. To this mixture, 1 ml of 2.5 N HCl was added, and the mixture was diluted with methanol to 50 ml. Then, the mixture was filtered though a 0.45-μm filter, and used as a HPLC sample. For the HPLC measurement, a fluorescence detector was used. The measurement was carried out under the following conditions.

### Column: ODS (Senshu Scientific Co., Ltd.)

Flow rate: 1 ml/min
Mobile phase: Acetonitrile:TFA=1:1
Injection amount: 2 μl
Detection: ex270, em330

FIG. 2 shows change in capsaicin concentration. As shown in FIG. 2, the decomposition of capsaicin in the capsicum oleoresin was apparently prevented by storing the capsicum oleoresin in the presence of water in the form of the composite material which was formed together with the phytosterol ester and the γ-cyclodextrin. Note that the ratio of capsaicin remaining after the enzymatic reaction for 60 minutes was 78.6% in Reference Example 2, 58.9% in Reference Comparative Example 2, and 2.0% in Reference Example, relative to that at the beginning of the shaking.

### Reference Example 3

Capsinoids extracted from “Natura” manufactured by AJINOMOTO CO., INC., were used.

To 0.70 parts by weight of a phytosterol ester heated to 70° C., 0.35 parts by weight of a fat and fatty oil containing the capsinoids was added, and dissolved therein. Meanwhile, 7.0 parts by weight of γ-cyclodextrin and 3.5 parts by weight of water were added to a mortar, and mixed with each other in a hot water bath at 70° C. to obtain a paste. To this paste, 1.05 parts by weight of the above-described oil phase in which the capsinoids were dissolved were added, and the mixture was kneaded in a hot water bath at 70° C. for 10 minutes. Thus, a composite material was prepared. Into 87.6 parts by weight of water, 11.55 parts by weight of the obtained composite material, 0.56 parts by weight of citric acid, and 0.27 parts by weight of trisodium citrate were dispersed, and the dispersion was stirred with a mixer for 30 seconds. Thus, a model beverage containing the composite material was prepared. The model beverage containing the composite material was heated up to 93° C., sterilized by being held at 90° C. for 3 minutes, and then packed into a pouch. Thereafter, the pouch was held in a constant-temperature water bath at 83° C. for 7 minutes to perform second sterilization.

### Reference Comparative Example 3-1

Capsinoids extracted from “Natura” manufactured by AJINOMOTO CO., INC., were used.

To 0.70 parts by weight of refined rapeseed oil heated to 70° C., 0.35 parts by weight of a fat and fatty oil containing the capsinoids was added, and dissolved therein. To 10.2 parts by weight of water, 0.33 parts by weight of an emulsifier (a polyglycerin fatty acid ester SWA-10D manufactured by Mitsubishi-Kagaku Foods Corporation), and 1.05 parts by weight of the above-described oil phase in which the capsinoids were dissolved were added, and the mixture was emulsified with a mixer. Thus, an emulsion was prepared. Into 87.6 parts by weight of water, 11.58 parts by weight of the obtained emulsion, 0.56 parts by weight of citric acid, and 0.27 parts by weight of trisodium citrate were dispersed, and the dispersion was stirred with a mixer for 30 seconds. Thus, an emulsion-containing model beverage was prepared. The emulsion-containing model beverage was heated up to 93° C., sterilized by being held at 90° C. for 3 minutes, and then packed into a pouch. Thereafter, the pouch was held in a constant-temperature water bath at 83° C. for 7 minutes to perform second sterilization.

### Reference Comparative Example 3-2

Capsinoids extracted from “Natura” manufactured by AJINOMOTO CO., INC., were used.

To 0.70 parts by weight of refined rapeseed oil heated to 70° C., 0.35 parts by weight of a fat and fatty oil containing the capsinoids were added, and dissolved therein. Meanwhile, 7.0 parts by weight of γ-cyclodextrin and 3.5 parts by weight of water were added to a mortar, and mixed with each other in a hot water bath at 70° C. to obtain a paste. To this paste, 1.05 parts by weight of the above-described oil phase in which the capsinoids were dissolved was added, and the mixture was kneaded in a hot water bath at 70° C. for 10 minutes. Thus, a composite material was prepared. Into 87.6 parts by weight of water, 11.55 parts by weight of the obtained composite material, 0.56 parts by weight of citric acid, 0.27 parts by weight of trisodium citrate were dispersed, and the dispersion was stirred with a mixer for 30 seconds. Thus, a model beverage containing the composite material was prepared. The model beverage containing the composite material was heated up to 93° C., and sterilized by being held at 90° C. for 3 minutes, and then packed into a pouch. Thereafter, the pouch was held in a constant-temperature water bath at 83° C. for 7 minutes to perform second sterilization.

TABLE 7

| | | Reference | Reference |
|---|---|---|---|
| Raw material (parts by | Reference | Comparative | Comparative |
| weight) | Example 3 | Example 3-1 | Example 3-2 |
| Fat and fatty acid containing | 0.35 | 0.35 | 0.35 |
| capsinoids | | | |
| (extracted from “Natura” | | | |
| manufactured by | | | |
| AJINOMOTO CO., INC.) | | | |
| Phytosterol ester | 0.70 | — | — |
| (San Sterol No. 3 | | | |
| manufactured by San-Ei Gen | | | |
| F. F. I., Inc.) | | | |
| Refined rapeseed oil | — | 0.70 | 0.70 |
| (manufactured by J-OIL | | | |
| MILLS, Inc.) | | | |
| γ-Cyclodextrin | 7.0 | — | 7.0 |
| Water | 3.5 | 10.2 | 3.5 |
| Emulsifier | — | 0.33 | — |
| (SWA-10D manufactured by | | | |
| Mitsubishi-Kagaku Foods | | | |
| Corporation) | | | |
| Citric acid | 0.56 | 0.56 | 0.56 |
| Trisodium citrate | 0.27 | 0.27 | 0.27 |
| Water | 87.6 | 87.6 | 87.6 |
| Total | 100 | 100 | 100 |

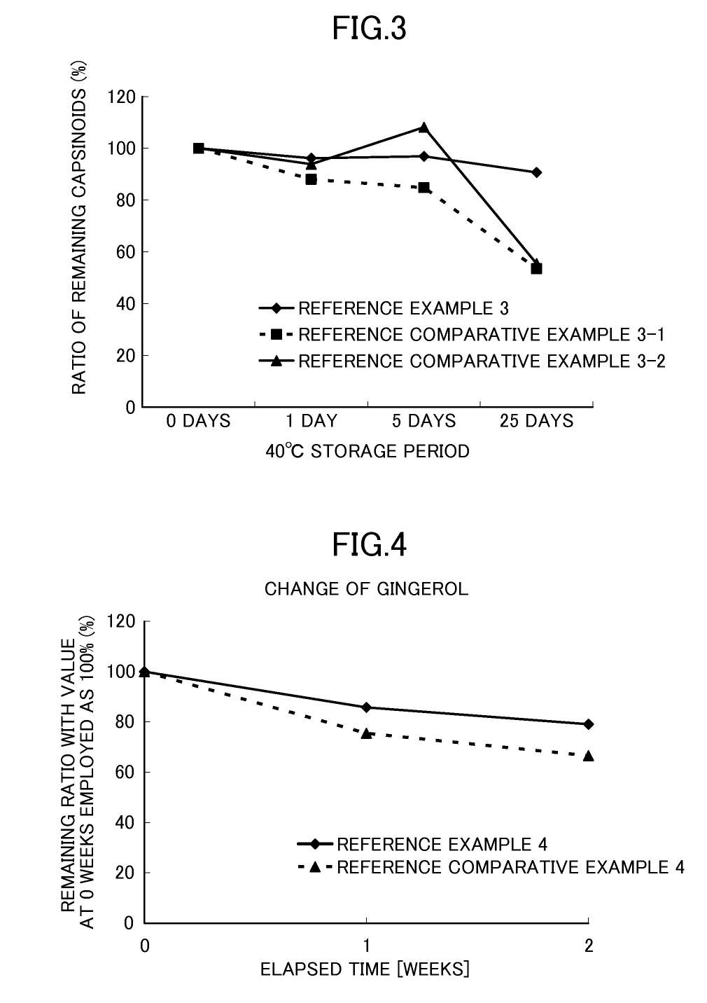

The model beverages prepared in Reference Example 3 and Reference Comparative Examples 3-1 and 3-2 were stored at 40° C. After certain periods of time had elapsed, the capsinoids in the samples were quantitatively determined by liquid chromatography. As the ratio of remaining capsinoids, values determined after the beverages were stored for 1 day, 5 days, and 25 days at 40° C. were represented by percentage, with a value of the capsinoids immediately after the beginning of the storage (0 days) being employed as 100%. FIG. 3 shows the results. As is apparent from FIG. 3, the decomposition of the capsinoids during the storage at 40° C. was more remarkably prevented in Reference Example 3 than in Reference Comparative Examples 3-1 and 3-2. From the results described above, it has been found that the present invention makes it possible to prevent the decomposition of capsinoids in the presence of water, and improve the stability thereof.

### Pretreatment Method for Liquid Chromatography

Regarding each of Reference Example 3 and Reference Comparative Example 3-2, 12.5 g of the model beverage was centrifuged (at 3000 rpm for 10 minutes), and then the supernatant was removed. To the deposit, 6 ml of DMSO (dimethyl sulfoxide) was added, and the mixture was ultrasonicated to dissolve the deposit. Moreover, the mixture was diluted with methanol to 25 ml, filtered through a 0.45-μm filter, and then used as a test liquid.

Regarding Reference Comparative Example 3-1, 5 g of the model beverage was sampled. The sample was diluted with methanol to 10 ml, filtered through a 0.45-μm filter, and then used as a test liquid.

### Measurement Conditions for Liquid Chromatography

A fluorescence detector was used.

### Column: Mightysil (250 mm, ø2.0)

Flow rate: 0.2 ml/min
Injection amount: 3 μl
Mobile phase: pH 3.3 TFA-water:Acetonitrile=20:80

### FLD Detection: EX270 EM330

### Reference Example 4

A supercritical ginger extract (gingerol: 24.8%, shogaol: 10.7%, Takasago International Corporation) was used as a ginger extract.

To 0.18 parts by weight of a phytosterol ester and 0.12 parts by weight of an edible fat and fatty oil, which were heated to 80° C., 0.015 parts by weight of the ginger extract was added and dissolved therein. Meanwhile, 1.093 parts by weight of γ-cyclodextrin and 1.093 parts by weight of water were mixed with each other by using a TK homomixer, while being heated to 80° C. To this mixture, 0.315 parts by weight of the above-described oil phase in which the ginger extract was dissolved was added. While continuously being heated to 80° C., the resultant mixture was stirred with a TK homomixer to conduct preliminary emulsification. After the preliminary emulsification, the mixture was passed through a high-pressure homogenizer (LAB1000 manufactured by SMT Co., Ltd., pressure: 100 MPa). Thus, a ginger extract-containing composite material was prepared. Into 97.08 parts by weight of water, 2.5 parts by weight of the obtained composite material, 0.3 parts by weight of citric acid, and 0.12 parts by weight of trisodium citrate were dispersed, and the dispersion was stirred for 30 seconds with a mixer. Thus, a model beverage containing the ginger extract composite material was prepared. The model beverage containing the ginger extract composite material was heated up to 93° C., sterilized by being held at 90° C. for 3 minutes, and packed into a pouch. Thereafter, the pouch was held in a constant-temperature water bath at 83° C. for 5 minutes to perform second sterilization. The gingerol component was 36.1 ppm, and the shogaol component was 15.4 ppm, in the prepared model beverage containing the ginger extract composite material.

### Reference Comparative Example 4

Here, an emulsion preparation obtained by emulsifying a ginger extract (gingerol: 1.79%, shogaol: 0.89%, Takasago International Corporation) was used.

Into 99.35 parts by weight of water, 0.23 parts by weight of the emulsion preparation, 0.3 parts by weight of citric acid, and 0.12 parts by weight of trisodium citrate were dispersed, and the dispersion was stirred with a mixer for 30 seconds. Thus, a model beverage containing the ginger extract emulsion preparation was prepared. The model beverage containing the ginger extract emulsion preparation was heated up to 93° C., sterilized by being held at 90° C. for 3 minutes, and then packed into a pouch. Thereafter, the pouch was held in a constant-temperature water bath at 83° C. for 5 minutes to perform a second sterilization. The gingerol component was 40.9 ppm, and the shogaol component was 16.2 ppm, in the prepared model beverage containing the ginger extract emulsion preparation.

TABLE 8

| | | Reference |
|---|---|---|
| | Reference | Comparative |
| Raw material (parts by weight) | Example 4 | Example 4 |
| Ginger extract | 0.015 | — |
| (Supercritical extract, gingerol: 24.8%, | | |
| shogaol: 10.7%, manufactured by Takasago | | |
| International Corporation) | | |
| Ginger extract | — | 0.23 |
| (Emulsion preparation, gingerol: 1.79%, | | |
| shogaol: 0.89%, manufactured by Takasago | | |
| International Corporation) | | |
| Phytosterol ester | 0.18 | — |
| (San Sterol No. 3 manufactured by San-Ei | | |
| Gen F. F. I., Inc.) | | |
| Edible fat and fatty oil | 0.12 | — |
| γ-Cyclodextrin | 1.093 | — |
| Water | 1.093 | — |
| Citric acid | 0.3 | 0.3 |
| Trisodium citrate | 0.12 | 0.12 |
| Water | 97.08 | 99.35 |
| Total | 100 | 100 |

The model beverages prepared in Reference Example 4 and Reference Comparative Example 4 were stored at 60° C. The gingerol and shogaol in the samples before the storage, after one-week storage, and after two-week storage were quantitatively determined by liquid chromatography. As the ratios of remaining gingerol and shogaol, the values after one-week storage and two-week storage were represented by percentage, with each of the values before the storage (0 weeks) being employed as 100%. FIGS. 4 and 5 show the results. As is apparent from FIGS. 4 and 5, the decomposition of gingerol and especially of shogaol was more prevented in Reference Example 4 than in Reference Comparative Example 4. From the results described above, it has been found that the present invention makes it possible to prevent the decomposition of a ginger extract in the presence of water, and to improve the stability thereof.

### Pretreatment Method for Liquid Chromatography

Regarding Reference Example 4, 25 g of the model beverage was centrifuged (at 3000 rpm, 10 minutes), and then the supernatant was removed. To the deposit, 3 ml of DMSO (dimethyl sulfoxide) was added, and the mixture was ultrasonicated to dissolve the deposit. Moreover, the mixture was diluted with methanol to 50 ml, filtered through a 0.45-μm filter, and then used as a test liquid.

Regarding Reference Comparative Example 4, 25 g of the model beverage was sampled. The sample was diluted with methanol to 50 ml, filtered through a 0.45-μm filter, and then used as a test liquid.

### Measurement Conditions for Liquid Chromatography

### UV: 282 nm

### Column: ODS C18 (Senshu Scientific Co., Ltd.)

Flow rate: 1.0 ml/min
Injection amount: 20 μl
Analysis time: 30 minutes
Mobile phase: Acetonitrile:Water:THF (tetrahydrofuran)=45:50:5

### Reference Example 5

A piperine powder (piperine content: 92% or more, Inabata Koryo Co., Ltd.) was used as a pepper extract.

To 0.18 parts by weight of a phytosterol ester and 0.12 parts by weight of an edible fat and fatty oil, which were heated to 80° C., 0.0064 parts by weight of the pepper extract was added, and dissolved therein. Meanwhile, 1.097 parts by weight of γ-cyclodextrin and 1.097 parts by weight of water were mixed with each other by using a TK homomixer, while being heated to 80° C. To this mixture, 0.3064 parts by weight of the above-described oil phase in which the pepper extract was dissolved was added. While continuously being heated to 80° C., the resultant mixture was stirred with a TK homomixer to conduct preliminary emulsification. After the preliminary emulsification, the mixture was passed through a high-pressure homogenizer (LAB1000 manufactured by SMT Co., Ltd., pressure: 100 MPa). Thus, a pepper extract-containing composite material was prepared. Into 97.08 parts by weight of water, 2.5 parts by weight of the obtained composite material, 0.3 parts by weight of citric acid, and 0.12 parts by weight of trisodium citrate were dispersed, and the dispersion was stirred with a mixer for 30 seconds. Thus, a model beverage containing the pepper extract composite material was prepared. The model beverage containing the pepper extract composite material was heated to 93° C., and sterilized by being held at 90° C. for 3 minutes, and then packed into a pouch. Thereafter, the pouch was held in a constant-temperature water bath at 83° C. for 5 minutes to conduct a second sterilization. The amount of piperine was 62 ppm in the model beverage containing the pepper extract composite material.

### Reference Comparative Example 5

Here, an extract of Piper longum of the Piperaceae family (content of piperines: 300 to 1400 ppm, Maruzen Pharmaceuticals Co., Ltd.) was used.

Into 99.43 parts by weight of water, 0.15 parts by weight of the pepper extract, 0.3 parts by weight of citric acid, and 0.12 parts by weight of trisodium citrate were dispersed, and the dispersion was stirred with a mixer for 30 seconds. Thus, a pepper extract-containing model beverage was prepared. The pepper extract-containing model beverage was heated to 93° C., sterilized by being held at 90° C. for 3 minutes, and then packed into a pouch. Thereafter, the pouch was held in a constant-temperature water bath at 83° C. for 5 minutes to perform a second sterilization. The amount of piperine was 0.25 ppm in the prepared pepper extract-containing model beverage.

TABLE 9

| | | Reference |
|---|---|---|
| | Reference | Comparative |
| Raw material (parts by weight) | Example 5 | Example 5 |
| Pepper extract | 0.0064 | — |
| (piperine powder, piperine content: 92% or | | |
| more, manufactured by Inabata Koryo Co., | | |
| Ltd.) | | |
| pepper extract | — | 0.15 |
| (extract of Piper longum of the Piperaceae | | |
| family, content of piperines: 300 to 1400 ppm, | | |
| manufactured by Maruzen Pharmaceuticals | | |
| Co., Ltd.) | | |
| Phytosterol ester | 0.18 | — |
| (San Sterol No. 3 manufactured by San-Ei | | |
| Gen F. F. I., Inc.) | | |
| Edible fat and fatty oil | 0.12 | — |
| γ-Cyclodextrin | 1.097 | — |
| Water | 1.097 | — |
| Citric acid | 0.3 | 0.3 |
| Trisodium citrate | 0.12 | 0.12 |
| Water | 97.08 | 99.43 |
| Total | 100 | 100 |

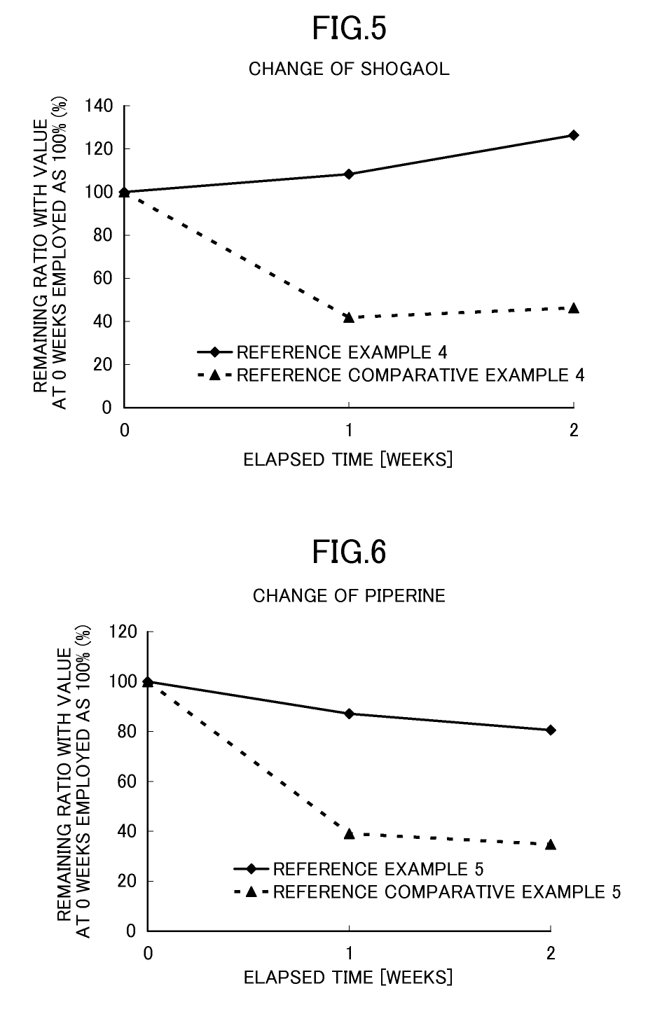

The model beverages prepared in Reference Example 5 and Reference Comparative Example 5 were stored at 60° C. Piperine in the samples before the storage, after one-week storage, and after two-week storage was quantitatively determined by liquid chromatography. As the ratio of remaining piperine, the values after one-week storage and two week storage were represented by percentage, with a value of piperine before the storage (0 weeks) being employed as 100%. FIG. 6 shows the results. As is apparent from FIG. 6, the decomposition of the piperine was more prevented in Reference Example 5 than in Reference Comparative Example 5. From the results described above, it has been found that the present invention makes it possible to prevent the decomposition of a pepper extract in the presence of water, and to improve the stability thereof.

### Pretreatment Method for Liquid Chromatography

Regarding Reference Example 5, 10 g of the model beverage was centrifuged (at 3000 rpm for 10 minutes), and then the supernatant was removed. To the deposit, 3 ml of DMSO (dimethyl sulfoxide) was added, and the mixture was ultrasonicated to dissolve the deposit. Moreover, the mixture was diluted with methanol to 50 ml, filtered through a 0.45-μm filter, and then used as a test liquid.

Regarding Reference Comparative Example 5, the sample was diluted with methanol, then filtered through a 0.45-μm filter, and used as a test liquid.

### Measurement Conditions for Liquid Chromatography

### UV: 343 nm

### Column: YMCPack ODS-A

Flow rate: 1.0 ml/min
Injection amount: 5 μl
Mobile phase: Acetonitrile:Water:THF (tetrahydrofuran)=45:55:7

### Reference Example 6

A deodorized fish oil “DHA-22HG” containing 22% or more of DHA (manufactured by Maruha Nichiro Foods, inc.) was used as an unsaturated fatty acid.

To 0.9 parts by weight of a phytosterol ester, 0.455 parts by weight of the deodorized fish oil containing DHA was added. The mixture was heated to 70° C. with string, and the deodorized fish oil was dissolved therein. Thus, a phytosterol ester in which the deodorized fish oil containing DHA was dissolved was prepared. Separately, 10 parts by weight of γ-cyclodextrin and 5 parts by weight of water (90° C.) were mixed with each other to prepare a mixture (paste). To the mixed paste, the phytosterol ester in which the deodorized fish oil containing DHA was dissolved was added. The mixture was kneaded for 10 minutes by using a mortar, while being heating to 70° C. Thus, a composite material was prepared. To the composite material, 82.895 parts by weight of water was added with mixing. Subsequently, 0.5 parts by weight of citric acid and 0.25 parts by weight of trisodium citrate were added thereto and mixed therewith. Furthermore, the resultant mixture was stirred with a homomixer at 5000 rpm for 2 minutes to obtain a homogeneous white liquid. The white liquid was heated to 93° C. with string, then packed into a colorless transparent glass container, and then cooled. Thus, a packed beverage was produced. Note that the pH of the beverage was 3.4.

### Reference Comparative Example 6-1

A deodorized fish oil “DHA-22HG” containing 22% or more of DHA (manufactured by Maruha Nichiro Foods, inc.) was used as an unsaturated fatty acid.

To 0.9 parts by weight of a phytosterol ester, 0.455 parts by weight of the deodorized fish oil containing DHA was added. The mixture was heated to 70° C. with string, and the deodorized fish oil containing DHA was dissolved therein. Thus, a phytosterol ester in which the deodorized fish oil containing DHA was dissolved was prepared. Separately, 0.5 parts by weight of an emulsifier was dissolved in 14.5 parts by weight of water (70° C.). To the emulsion, the phytosterol ester in which the deodorized fish oil containing DHA was dissolved was added, and the mixture was stirred with a homomixer at 5000 rpm for 10 minutes. Thus, an emulsion was prepared. To the emulsion, 82.895 parts by weight of water was added with mixing, and subsequently 0.5 parts by weight of citric acid and 0.25 parts by weight of trisodium citrate were added thereto and mixed therewith. Then, the mixture was heated up to 93° C. with string, packed into a colorless transparent glass container, and then cooled. Thus, a packed beverage was produced. Note that the pH of the beverage was 3.4.

### Reference Comparative Example 6-2

A deodorized fish oil “DHA-22HG” containing 22% or more of DHA (manufactured by Maruha Nichiro Foods, inc.) was used as an unsaturated fatty acid.

To 0.9 parts by weight of refined rapeseed oil, 0.455 parts by weight of the deodorized fish oil containing DHA was added. The mixture was heated to 70° C. with string, and the deodorized fish oil containing DHA was dissolved therein. Thus, refined rapeseed oil in which the deodorized fish oil containing DHA was dissolved was prepared. Separately, 0.5 parts by weight of an emulsifier was dissolved in 14.5 parts by weight of water (70° C.). To the emulsion, the refined rapeseed oil in which the deodorized fish oil containing DHA was dissolved was added, and the resultant mixture was stirred with a homomixer at 5000 rpm for 10 minutes. Thus, an emulsion was prepared. To the emulsion, 82.895 parts by weight of water was added with mixing, and subsequently 0.5 parts by weight of citric acid and 0.25 parts by weight of trisodium citrate were added thereto and mixed therewith. Thereafter, the mixture was heated to 93° C. with string, then packed into a colorless transparent glass container, and then cooled. Thus, a packed beverage was produced. Note that the pH of the beverage was 3.4.

### (Evaluation of Beverages)

The packed beverages were placed in a thermostatic chamber (“SANYO GROWTH CABINET”, temperature: 25° C., illuminance: 10000 lx), and stored for 6 days. After the storage, the beverages were subjected to sensory evaluation in terms of the odor (fishy odor). The blending ratios and the results of the sensory evaluation are shown in the following Table 10. From these results, it has been found that the present invention makes it possible to prevent the deterioration of the deodorized fish oil containing DHA.

TABLE 10

| | | | Reference | |
|---|---|---|---|---|
| | | Reference | Comparative | Reference |
| | | Example 6 | Example 6-1 | Example 6-2 |
| Blended | Deodorized fish oil | 0.455 | 0.455 | 0.455 |
| ratio | containing DHA | | | |
| (parts | (“DHA-22HG,” | | | |
| by | Maruha Nichiro | | | |
| weight) | Foods, inc.) | | | |
| | Phytosterol ester | 0.9 | 0.9 | — |
| | (“San Sterol No. 3,” | | | |
| | San-Ei Gen F. F. I., | | | |
| | Inc.) | | | |
| | Refined rapeseed oil | — | — | 0.9 |
| | (manufactured by J- | | | |
| | OIL MILLS, Inc.) | | | |
| | γ-Cyclodextrin | 10 | — | — |
| | Emulsifier (“Ryoto | — | 0.5 | 0.5 |
| | Ester SWA-10D,” | | | |
| | Mitsubishi-Kagaku | | | |
| | Foods Corporation) | | | |
| | Water | 5 | 14.5 | 14.5 |
| | Citric acid (anhydrous) | 0.5 | 0.5 | 0.5 |
| | Trisodium citrate | 0.25 | 0.25 | 0.25 |
| | Water | 82.895 | 82.895 | 82.895 |
| | Total | 100 | 100 | 100 |

| | | | |
|---|---|---|---|
| Sensory evaluation (fishy odor) | Almost no | Strong odor | Strong odor |
| | odor was | was noticed | was noticed |
| | noticed | | |

### Reference Example 7

A deodorized fish oil “DHA-22HG” containing 22% or more of DHA (manufactured by Maruha Nichiro Foods, inc.) was used as an unsaturated fatty acid.

To 0.9 parts by weight of a phytosterol ester, 0.455 parts by weight of the deodorized fish oil containing DHA was added. The mixture was heated to 70° C. with string, and the deodorized fish oil containing DHA was dissolved therein. Thus, a phytosterol ester in which the deodorized fish oil containing DHA was dissolved was prepared. Separately, 10 parts by weight of γ-cyclodextrin and 5 parts by weight of water (90° C.) were mixed with each other to prepare a mixture (paste). To the mixed paste, the phytosterol ester in which the deodorized fish oil containing DHA was dissolved was added. The mixture was kneaded for 10 minutes by using a mortar, while being heated to 70° C. Thus, a composite material was prepared. To the composite material, 82.895 parts by weight of water was added with mixing, and subsequently 0.5 parts by weight of citric acid and 0.25 parts by weight of trisodium citrate were added thereto and mixed therewith. Furthermore, the resultant mixture was stirred with a homomixer at 5000 rpm for 2 minutes to obtain a homogeneous white liquid. The white liquid was heated to 93° C. with string, then packed into a colorless transparent glass container, and then cooled. Thus, a packed beverage was produced. Note that the pH of the beverage was 3.4.

### Reference Comparative Example 7

A deodorized fish oil “DHA-22HG” containing 22% or more of DHA (manufactured by Maruha Nichiro Foods, inc.) was used as an unsaturated fatty acid.

A mixture (paste) was prepared by mixing 10 parts by weight of γ-cyclodextrin and 5 parts by weight of water (90° C.) with each other. To the mixed paste, the deodorized fish oil containing DHA was added. The mixture was kneaded for 10 minutes by using a mortar, while being heated to 70° C. Thus, a composite material was prepared. To the composite material, 83.795 parts by weight of water was added with mixing, and subsequently 0.5 parts by weight of citric acid and 0.25 parts by weight of trisodium citrate were added thereto and mixed therewith. Furthermore, the resultant mixture was stirred with a homomixer at 5000 rpm for 2 minutes to obtain a homogeneous white liquid. The white liquid was heated up to 93° C. with string, then packed into a colorless transparent glass container, and then cooled. Thus, a packed beverage was produced. Note that the pH of the beverage was 3.4.

### (Evaluation of Beverages)

The packed beverages were placed in a thermostatic chamber (“SANYO GROWTH CABINET”, temperature: 25° C., illuminance: 10000 lx), and stored for 6 days. After the storage, the beverages were subjected to sensory evaluation in terms of the odor (fishy odor). Furthermore, the peroxide values thereof were measured (testing method: an acetic acid-isooctane method). The blending ratios and the results of the sensory evaluation are shown in the following Table 11. From these results, it has been found that the present invention makes it possible to prevent the deterioration of the deodorized fish oil containing DHA.

TABLE 11

| | | | Reference |
|---|---|---|---|
| | | Reference | Comparative |
| | | Example 7 | Example 7 |
| Blended ratio | Deodorized fish oil | 0.455 | 0.455 |
| (parts by weight) | containing DHA | | |
| | (“DHA-22HG,” Maruha | | |
| | Nichiro Foods, inc.) | | |
| | Phytosterol ester | 0.9 | — |
| | (“San Sterol No. 3,” San-Ei | | |
| | Gen F. F. I., Inc.) | | |
| | γ-Cyclodextrin | 10 | 10 |
| | Water | 5 | 5 |
| | Citric acid (anhydrous) | 0.5 | 0.5 |
| | Trisodium citrate | 0.25 | 0.25 |
| | Water | 82.895 | 83.795 |
| | Total | 100 | 100 |

| | | |
|---|---|---|
| Sensory evaluation (fishy odor) | Almost no | Strong odor |
| | odor was | was noticed |
| | noticed | |
| Peroxide value (meq/kg) | 58 | 618 |

Hereinafter, Examples of Japanese Patent Application No. 2008-328263 are described as Reference Examples of the food or active pharmaceutical ingredient (hydrophilic component) to which the present invention can be applied.

### Reference Example 8

To 6.19 parts by weight of a phytosterol ester melted by heating to 60° C., 0.06 parts by weight of a sucrose fatty acid ester was added, and mixed therewith. The mixture was stirred with a homogenizer at 7000 rpm for 30 seconds to obtain a homogeneous dispersion. Next, 0.69 parts of caffeine was added to the phytosterol ester in which the sucrose fatty acid ester was dispersed. Then, the mixture was again stirred with a homogenizer at 7000 rpm for 1 minute to obtain a homogeneous dispersion. Thus, a first mixture was prepared. Separately from the first mixture, 55.83 parts by weight of γ-cyclodextrin and 37.23 parts by weight of water (60° C.) were added to a mortar, and mixed with each other by using a pestle. Thus, a second mixture was prepared as a paste. The first mixture and the second mixture were kneaded in a mortar for 10 minutes to thereby form a composite material. The blended amounts (g) in Reference Example 8 are shown in the following Table 12.

### Reference Comparative Example 8

To a mortar, 55.83 parts by weight of γ-cyclodextrin and 43.48 parts by weight of water (60° C.) were added, and were mixed with each other by using a pestle. Thus, a mixture was prepared as a paste. To the mixture, 0.69 parts by weight of caffeine was added, and the resultant mixture was kneaded in hot water (60° C.) for 10 minutes. Thus, a composite material was formed. The blended amounts (g) in Reference Comparative Example 8 are shown in the following Table 12.

TABLE 12

| | | Reference |
|---|---|---|
| | Reference | Comparative |
| Raw material | Example 8 | Example 8 |
| Caffeine | 0.69 g | 0.69 g |
| Phytosterol ester | 6.19 g | — |
| (San Sterol No. 3 manufactured by San-Ei | | |
| Gen F. F. I., Inc.) | | |
| Sucrose fatty acid ester | 0.06 g | — |
| γ-Cyclodextrin | 55.83 g | 55.83 g |
| Water | 37.23 g | 43.48 g |
| Total | 100.00 g | 100.00 g |

### (Evaluation of Reduction of Bitter Taste of Caffeine)

At the ratios shown in the following Table 13, water was added to and mixed with each of the composite materials obtained in Reference Example 8 and Reference Comparative Example 8, and then sensory evaluation was conducted. In addition, comparison was made with a mixture of caffeine and water being employed as Reference Example.

The bitter taste was apparently more reduced in Reference Example 8 than in Reference Comparative Example 8 and Reference Example. The bitter taste was more reduced in Reference Comparative Example 8 than in Reference Example, but the bitter taste was still noticed in Reference Comparative Example 8.

TABLE 13

| | | Reference | |
|---|---|---|---|
| | Reference | Comparative | Reference |
| Raw material | Example 8 | Example 8 | Example |
| Composite material | 14.5 g | 14.5 g | — |
| Caffeine | — | — | 0.1 g |
| Water | 85.5 g | 85.5 g | 99.9 g |
| Total | 100.0 g | 100.0 g | 100.0 g |

* Caffeine concentration in each of Reference Example 8, Reference Comparative Example 8, and Reference Example: 0.1%

### Reference Example 9

To 6.21 parts by weight of a phytosterol ester melted by heating to 60° C., 0.06 parts by weight of a sucrose fatty acid ester was added, and mixed therewith. The mixture was stirred with a homogenizer at 7000 rpm for 30 seconds to obtain a homogeneous dispersion. Next, 0.62 parts by weight of vitamin B1 was added to the phytosterol ester in which the sucrose fatty acid ester was dispersed. Then, the mixture was again stirred with a homogenizer at 7000 rpm for 1 minute to obtain a homogeneous dispersion. Thus, a first mixture was prepared. Separately from the first mixture, 55.87 parts by weight of γ-cyclodextrin and 37.24 parts by weight of water (60° C.) were added to a mortar, and mixed with each other by using a pestle. Thus, a second mixture was prepared as a paste. The first mixture and the second mixture were kneaded in a mortar for 10 minutes to thereby form a composite material. The blended amounts (g) in Reference Example 9 are shown in the following Table 14.

### Reference Comparative Example 9

To a mortar, 55.87 parts by weight of γ-cyclodextrin and 43.51 parts by weight of water (60° C.) were added, and mixed with each other by using a pestle. Thus, a mixture was prepared as a paste. To the mixture, 0.62 parts by weight of vitamin B1 was added, and the mixture was kneaded in a hot water (60° C.) for 10 minutes. Thus, a composite material was formed. The blended amounts (g) in Reference Comparative Example 9 are shown in the following Table 14.

TABLE 14

| | | Reference |
|---|---|---|
| | Reference | Comparative |
| Raw material | Example 9 | Example 8 |
| Vitamin B1 | 0.62 g | 0.62 g |
| Phytosterol ester | 6.21 g | — |
| (San Sterol No. 3 manufactured by San-Ei | | |
| Gen F. F. I., Inc.) | | |
| Sucrose fatty acid ester | 0.06 g | — |
| γ-Cyclodextrin | 55.87 g | 55.87 g |
| Water | 37.24 g | 43.51 g |
| Total | 100.00 g | 100.00 g |

### (Evaluation of Reduction of Bitter Taste of Vitamin B1)

At the ratios shown in the following Table 15, a 0.1% citric acid solution was added to and mixed with each of the composite materials obtained in Reference Example 9 and Reference Comparative Example 9, and then sensory evaluation was conducted. In addition, a mixture obtained by dissolving vitamin B1 in a 0.1% citric acid solution was employed as Reference Example.

The bitter taste was apparently more reduced in Reference Example 9 than in Reference Comparative Example 9 and Reference Example. The bitter taste was slightly reduced in Reference Comparative Example 9 as compared with Reference Example, but the strong bitter taste was still noticed in Comparative Example 9.

TABLE 15

| | | Reference | |
|---|---|---|---|
| | Reference | Comparative | Reference |
| Raw material | Example 9 | Example 9 | Example |
| Composite material | 2.415 g | 2.415 g | — |
| Vitamin B1 | — | — | 0.015 g |
| 0.1% citric acid solution | 97.585 g | 97.585 g | 99.985 g |
| Total | 100.0 g | 100.0 g | 100.0 g |

* Vitamin B1 concentration in each of Reference Example 9, Reference Comparative Example 9, and Reference Example: 0.015%

### Reference Example 10

To 9.0 parts by weight of a phytosterol ester melted by heating to 60° C., 2.1 parts by weight of a glycerin fatty acid ester and 2.3 parts by weight of red beet pigment were added, and the mixture was stirred with a homogenizer at 7000 rpm for 1 minute to obtain a homogeneous dispersion. Thus, a first mixture was prepared. Separately from the first mixture, 43.3 parts by weight of γ-cyclodextrin and 43.3 parts by weight of water (60° C.) were added to a mortar, and mixed with each other by using a pestle. Thus, a second mixture was prepared as a paste. The first mixture and the second mixture were kneaded in a mortar for 10 minutes. Thus, a composite material having a pink color tone was formed. The blended amounts (g) in Reference Example 10 are shown in the following Table 16.

### Reference Comparative Example 10

To a mortar, 43.3 parts by weight of γ-cyclodextrin and 54.4 parts by weight of water (60° C.) were added, and mixed with each other by using a pestle. Thus, a mixture was prepared as a paste. To this mixture, 2.3 parts by weight of red beet pigment was added, and the resultant mixture was kneaded in hot water (60° C.) for 10 minutes. Thus, a composite material having a pink color tone was formed.

The blended amounts (g) in Reference Comparative Example 10 are shown in the following Table 16.

TABLE 16

| | | Reference |
|---|---|---|
| | Reference | Comparative |
| Raw material | Example 10 | Example 10 |
| Red beet pigment | 2.3 g | 2.3 g |
| (containing betanin and isobetanin) | | |
| Phytosterol ester | 9.0 g | — |
| (San Sterol No. 3 manufactured by San-Ei | | |
| Gen F. F. I., Inc.) | | |
| Glycerin fatty acid ester | 2.1 g | — |
| γ-Cyclodextrin | 43.3 g | 43.3 g |
| Water | 43.3 g | 54.4 g |
| Total | 100.0 g | 100.0 g |

### (Evaluation of Color Tone Stability of Red Beet Pigment)

At the ratios shown in the following Table 17, water was added to and mixed with each of the composite materials obtained in Reference Example 10 and Reference Comparative Example 10, and then each composite material in water was packed and sealed in a transparent pouch. In addition, as Reference Example, red beet pigment and water were mixed with each other at the ratio shown in Table 17, and were packed and sealed in a transparent pouch. These pouches were stored in a thermostatic chamber at 65° C. for 14 hours, and the change in color tone before and after storage was visually evaluated.

The pink color tone of Reference Example 10 was kept even after the storage. In contrast, the pink color tone of each of Reference Comparative Example 10 and Reference Example was completely lost, and these liquids had appearances which were transparent but slightly mixed with an orange color.

TABLE 17

| | | Reference | |
|---|---|---|---|
| | Reference | Comparative | Reference |
| | Example 10 | Example 10 | Example |
| Composite material | 14.1 g | 14.1 g | — |
| Red beet pigment | — | — | 0.32 g |
| Water | 85.9 g | 85.9 g | 99.68 g |
| Total | 100.0 g | 100.0 g | 100.0 g |

* Red beet pigment concentration in each of Reference Example 10, Reference Comparative Example 10, and Reference Example: 0.32%

1. A composite material comprising a food or active pharmaceutical ingredient, a phytosterol ester, a medium-chain fatty acid triacylglyceride, and a cyclodextrin.

2. The composite material according to claim 1, which is obtained by mixing the food or active pharmaceutical ingredient, the phytosterol ester, the medium-chain fatty acid triacylglyceride, and the cyclodextrin with each other in the presence of water.

3. The composite material according to claim 1, wherein the food or active pharmaceutical ingredient is a lipophilic component.

4. The composite material according to claim 3, wherein the lipophilic component is a pungent component or a bitter component.

5. The composite material according to claim 1, wherein the food or active pharmaceutical ingredient is a hydrophilic component surface-treated with a surfactant.

6. A composition comprising the composite material according to claim 1.

7. A liquid composition comprising:
the composite material according to claim 1;
water; and
a thickener,
wherein the composite material is in a water-dispersed form.

8. A method for producing a composite material comprising a food or active pharmaceutical ingredient, a phytosterol ester, a medium-chain fatty acid triacylglyceride, and a cyclodextrin, the method comprising mixing the food or active pharmaceutical ingredient, the phytosterol ester, the medium-chain fatty acid triacylglyceride, and the cyclodextrin with each other in the presence of water, to form the composite material.

9. The method for producing a composite material according to claim 8, further comprising:
dissolving the food or active pharmaceutical ingredient in the phytosterol ester and the medium-chain fatty acid triacylglyceride;
preparing a mixture containing the cyclodextrin and the water; and
mixing the phytosterol ester and the medium-chain fatty acid triacylglyceride, in which the food or active pharmaceutical ingredient is dissolved, with the said mixture, to form the composite material.
